(19) United States
(12) Patent Application Publication
(10) Pub. No.: US 2004/0033584 A1
(43) Pub. Date: Feb. 19, 2004

(54) Therapeutic use of particles displaying pathogen-specific binding moieties

(76) Inventor: Joshua Lederberg, New York, NY (US)

Correspondence Address: PENNIE AND EDMONDS, 1155 AVENUE OF THE AMERICAS, NEW YORK, NY, 100362711

(21) Appl. No.: 10/168,855

(22) PCT Filed: Dec. 21, 2000
(86) PCT No.: PCT/US00/35076

(51) Int. Cl. A61K 7/46; C12Q 1/70; C12N 7/00; C12N 7/01; C07K 1/00; C07K 14/00; C07K 17/00

(52) U.S. Cl. 435/235.1; 512/2; 530/350

(57) ABSTRACT

The present invention relates to the therapeutic use of particles which are efficiently removed from the circulation, the said particles display pathogen-specific binding moieties to chaperone the clearance of potentially injurious substances from a vertebrate's circulation. The invention is also drawn to methods for treating, diagnosing, or screening for diseases and disorders associated with the presence of the pathogens. The present invention also relates to pharmaceutical compositions and diagnostic kits containing the therapeutic particle which displays the pathogen binding moiety of the invention.

### 1. FIELD OF THE INVENTION

The present invention relates to the therapeutic use of particles which are cleared rapidly from the circulation, which is believed to occur by an intrinsic ability to be recognized and phagocytosed by cells of the Reticuloendothelial System (RES); the said particles display pathogen-specific binding moieties to enhance the clearance of potentially injurious substances from a vertebrate's circulation. The invention also relates to methods for treating, diagnosing, or screening for diseases and disorders associated with the presence of the pathogens. The present invention also relates to pharmaceutical compositions and diagnostic kits containing the therapeutic particle of the invention which displays the pathogen binding moiety.

### 2. BACKGROUND OF THE INVENTION

The fixed tissue phagocytes of the liver and spleen are primarily responsible for the clearance of circulating pathogens. These pathogens include bacteria, viruses, toxins, immune complexes, drugs, or anything that is present in the circulation and is detrimental to the health of a host vertebrate. Failure of the immune system to effectively remove the pathogens from the vertebrate circulation can lead to traumatic and hypovolemic shock (Altura and Hershey, 1968; Am. J. Physiol. 215:1414-9).

The clearance of pathogens from the circulation involves the binding of the pathogen with a receptor on a phagocyte and the subsequent removal of the pathogen from the circulation by endocytosis into the phagocyte leading to the pathogen's destruction by chemical and proteolytic degradation. The pathogen's rate and efficiency of removal from the circulation is dependent upon multiple factors including the number of fixed tissue phagocytes present in the organism, the number of appropriate receptors on the fixed tissue phagocytes, the serum concentration of opsonins, the affinity of the receptor for the pathogen, and the concentration of the pathogens (Reichard and Filkins, 1984, The Reticuloendothelial System; A Comprehensive Treatise, pp. 73-101 (Plenum Press)).

Serum opsonins, such as antibodies or complement enhance the clearance of a pathogen by binding to the pathogen and coating it so that it is more readily recognized by receptors on phagocytes. For example, the complement factor C3b clears pathogens by directing contact of the pathogen to the CR1 receptor expressed on erythrocytes, which then directs the particle to the RES for clearance. When C3b is pre-coated onto agarose beads, the beads are cleared more rapidly and efficiently from the circulation than uncoated beads (Johnson et al., 1983, Scand J Immunol 17:403).

Any moiety that can bind an antigen and is itself recognized by immune cells can serve as an opsonin. A significant limitation on the rate of clearance of pathogens from the circulation is low concentration of opsonins in the serum. The low number of opsonins relative to the number of pathogens present in the blood stream allows many of the particles to escape prompt and efficient clearance (Reichard and Filkins, 1984, The Reticuloendothelial System; A Comprehensive Treatise, pp. 73-101 (Plenum Press)).

### 2.1 THERAPEUTIC ANTIBODY PRODUCTION

The development of monoclonal antibody technology, first disclosed by Kohler and Milstein (1975, Nature 256:495-497), has allowed the generation of a nearly unlimited supply of antibodies of precise and reproducible specificity. The Kohler and Milstein procedure involves the fusion of spleen cells obtained from an immunized animal, with an immortal myeloma cell line which results in a population of hybridoma cells, which will include a hybridoma that produces an antibody of the desired specificity. The hybridoma which produces an antibody having the requisite specificity is then selected, or ‘cloned’, from this population of hybridomas using conventional techniques such as enzyme linked immunosorbent assays (ELISA).

Additional approaches to generating antibodies useful for therapeutic uses have been developed as an alternative to the laborious immunization procedure mentioned above. One approach entails cloning a sub-library of genes that encode an antibody in frame with phage structural proteins, then inserting these recombinant genes into bacteriophage, which will express the antibody-structural fusion protein on the virus surfaces as described in Clackson et al., 1991, Nature 352:624; Marks et al., 1992, J. Mol. Biol. 222:581; Zebedee et al., 1992, Proc. Natl. Acad. Sci. USA 39:3175; Gram et al., 1992, Proc. Natl. Acad. Sci. USA 89:3576. However, the production of an antibody that binds a pathogen of interest does not always result in a therapeutically effective antibody.

Taylor et al. (U.S. Pat. Nos. 5,487,890, 5,470,570 and 5,879,679) have shown that by attaching an antibody specific to a pathogen to a second antibody specific to the CR1 receptor, thus creating a heteropolymer, a vascular pathogen can be neutralized in the vertebrate circulation. This method of treating a subject is hindered where the subject is anemic or has a decreased number of CR1 receptors.

### 2.2 DEVELOPMENT OF THERAPEUTIC BINDING MOIETIES

Numerous techniques have been developed which identify potential binding moieties to pathogens in the hopes that these binding moieties will have utility as a therapeutic agent against the pathogen, for example combinatorial chemistry, or phage display libraries from which peptides or proteins are identified for potential use.

Combinatorial chemistry can be used to identify binding moieties. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, H., 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest. The fingerprints are compared with fingerprints obtained from other compounds known to react with the protein of interest to predict whether the library compound might similarly react. For example, rather than testing every ligand in a large library for interaction with a known receptor that is associated with a particular pharmacological activity (for example, antihistaminic or anticholinergic activity), only those ligands having a fingerprint similar to other compounds known to have that activity would be tested. (See, e.g., Kauvar, L. M. et al., 1995 Chemistry and Biology 2:107-1 18; Kauvar. L. M., 1995, Affinity fingerprinting, Pharmaceutical Manufacturing International. 8:25-28; and Kauvar, L. M., 1995 Toxic-Chemical Detection by Pattern Recognition in New Frontiers in Agrochemical Immunoassay, D. Kurtz. L. Stanker and J. H. Skerritt. Editors, AOAC: Washington, D.C., 305-312). The binding moiety need only contact the pathogen to be cleared from the circulation, i.e. the methods and compositions of the present invention do not require the identification of a biological function for the binding moiety.

Phage display has-been adapted to screen for peptides and antibodies that bind to a target substrate. Various different bacteriophage and methods of expressing the test peptides and antibodies have been reported. For example, M13 has been extensively characterized and it is known that the viral capsid protein of M13, protein III (pIII), is responsible for infection of bacteria. Several investigators have determined from mutational analysis that the 406 amino acid long pIII capsid protein has two domains. The C-terminus anchors the protein to the viral coat, while portions of the N-terminus of pIII are essential for interaction with the E. coli pilin protein (Crissman, J. W. and Smith, G. P., 1984, Virology 132:445-455). Although the N-terminus of the pIII protein has been shown to be necessary for viral infection, the extreme N-terminus of the mature protein does tolerate alterations. In 1985, George Smith published experiments reporting the use of the pIII protein of bacteriophage M13 as an experimental system for expressing a heterologous protein on the viral coat surface (Smith, G. P., 1985, Science 228:1315-1317). It was later recognized, independently by two groups, that the M13 phage pIII gene display system could be a useful one for mapping antibody epitopes (De la Cruz, V., et al., 1988, J. Biol. Chem. 263:4318-4322; Parmley, S. F. and Smith, G. P., 1988, Gene 73:305-318).

A significant weakness of the phage display and combinatorial chemistry techniques is that although the identified binding moiety may interact with the pathogen, there is no guarantee the binding moiety will have a therapeutic utility. For example, a binding moiety derived from the foregoing techniques will rarely direct the immune system to attack the pathogen and clear it from the circulation as would naturally occurring opsonins such as antibodies or complement. Alternatively, there is no reasonable expectation that the said binding moiety will interfere with the normal replication of the pathogen in the circulation, thereby therapeutically treating the subject by blocking the growth or perpetuation of the pathogen. Therefore, a need exists for a method of treating a subject with a pathogen-specific binding moiety such that upon contacting the pathogen, the complex is efficiently cleared from the circulation.

Citation of a reference herein shall not be construed as an admission that such is prior art to the present invention.

### 3. SUMMARY OF THE INVENTION

The present invention provides for the therapeutic use of particles which are cleared rapidly from the circulation, wherein the said particles display pathogen-specific binding moieties, in which the binding-moiety is not endogenous to the particle, to enhance the clearance of pathogenic agents which are potentially injurious substances, from a vertebrate's circulation. While not meaning to be bound by any mechanism, we suggest that the rapid clearance occurs by an intrinsic ability of the particle to be recognized and phagocytosed by cells of the reticuloendothelial system (RES). The invention also relates to methods for treating, diagnosing, or screening for diseases and disorders associated with the presence of the pathogens. The present invention also relates to pharmaceutical compositions and diagnostic kits containing the therapeutic particle which displays the pathogen binding moiety of the invention.

The invention further provides methods of producing a therapeutic pathogen-specific particle of the invention by phage display, combinatorial chemistry or other similar techniques known to those of ordinary skill in the art.

The present invention also provides methods for producing the pathogen-specific particles of the invention, for example, by recombinant protein expression or chemical crosslinking.

The present invention also provides for a method of treating a vertebrate having a disease or disorder associated with the presence of a pathogen comprising administering to the vertebrate a therapeutically effective dose of a particle bound to a binding moiety that specifically binds the pathogen, said binding moiety not being endogenous to said particle.

The invention further provides methods of treatment or prevention using the pathogen-specific particles of the invention. For example, particles of the invention that contain a binding moiety specific for an infectious disease agent or other pathogen can be used in the treatment or prevention of the infectious or pathogenic disease associated with the infectious or pathogenic disease agent.

The invention further provides, in a preferred embodiment, a particle that is a bacteriophage bound to a pathogen-specific binding moiety, which enhances clearance of the pathogen from the blood for therapeutic effect. The binding moiety in specific embodiments is a receptor or binding fragment thereof for a ligand of the pathogen, or, preferably, is an antibody or antibody fragment specific for the pathogen. The bacteriophage is covalently bound to the antibody or fragment either by expression of a recombinant fusion protein on its surface containing the pathogen-specific binding moiety, or by chemical crosslinking the pathogen-specific moiety to the bacteriophage.

The invention also provides for kits containing instructions for the methods of the present invention as well as pharmaceutical compositions containing the particles bound to the pathogen binding moiety.

The pathogen-specific particles of the invention can also be used prophylactically to prevent disease or disorder caused by a pathogen.

### 4. DETAILED DESCRIPTION OF THE INVENTION

The present invention provides for the therapeutic use of particles which are cleared rapidly from the circulation, wherein the said particles display pathogen-specific binding moieties, to enhance the clearance of pathogenic agents which are potentially injurious substances, from a vertebrate's circulation. While not meaning to be bound by any mechanism, the rapid clearance occurs by an intrinsic ability of the particle to be recognized and phagocytosed by cells of the reticuloendothelial system (RES). the invention also relates to methods for treating, diagnosing, or screening for diseases and disorders associated with the presence of the pathogens. The present invention also relates to pharmaceutical compositions and diagnostic kits containing the therapeutic particle which displays the pathogen binding moiety of the invention.

The particles of the present invention include but are not limited to denatured proteins, insoluble particles, metal oxides, suspensions of colloidal carbon particles, ‘ghost’ red blood cells (RBCs), empty viral capsids, bacteria, liposomes, cell fragments, or bacteriophage linked to a pathogen-specific binding moiety. Upon administration to a subject of the pathogen-specific particle of the invention, normal vascular clearance of the pathogen is assisted by opsonization with the particle which then directs the immune system to eliminate the pathogen. As will be apparent to the skill artisan, the term “particle” does not include intact red blood cells.

In a specific embodiment, the binding moiety is not endogenous to the particle. Thus, for example, the binding moiety is not found in nature as part of the particle. Thus, for example, according to the invention, a non-endogenous binding moiety may be cross-linked or otherwise covalently bound to the particle, including but not limited to expressing the binding moiety as a fusion protein of the particle.

Covalent binding of a pathogen-specific binding moiety to a particle of the present invention can significantly increase the efficiency of clearance of the pathogen from the circulation by the RES. In a specific embodiment, a particle of the invention is a bacteriophage that is cleared rapidly from the circulation (e.g., 90% clearance in 48 hours, preferably 24 hours) bound covalently to an antibody or antibody fragment specific for a pathogen.

The particle with the pathogen binding moiety of the invention is preferably up to 10,000 times or more smaller in mass than human erythrocytes, and therefore readily diffusible in the circulatory system. Furthermore, the pathogen-specific binding moiety linked to the particle can be any antibody, protein, ligand or chemical moiety, or binding portion/fragment thereof, which exhibits binding to the pathogen sought to be removed from the circulation. The binding moiety is preferably covalently bound to the particle.

### 4.1 RES CLEARED PARTICLES

The particles of the present invention can be denatured proteins (e.g., human serum albumin (Benacerraf et al., 1957 Brit. J. Exp. Path, 38:35)), insoluble particles (e.g., carbon black, silica, silicon dioxide), metal oxides (e.g., titanium oxides, iron oxides), and India ink (i.e., suspension of colloidal carbon particles) (described in Reichard and Filkins, 1984, The Reticuloendothelial System; A Comprehensive Treatise, pp. 73-101 (Plenum Press), and references therein). The particles of the present invention also may be red blood cells (RBCs) that have been purged of their cytoplasm, known as ‘Ghost’ RBCs, bacteria (as bacteria are cleared by the RES; see, e.g., Benacerraf and Miescher, 1960, Ann NY Acad Sci, 88:184-195), cell fragments, liposomes, bacteriophage, bacteriophage fragments, and viral capsids devoid of the viral nucleic acids (e.g., hepatitis B virus surface antigen particles), etc.

In a preferred embodiment of the present invention, bacteriophage are utilized as the particle which is cleared by the RES. Bacteriophage have monodispersity in the circulation and are cleared by the RES; furthermore, bacteriophage can be selected in vivo or in vitro for decreased kinetics of clearance (Merril et al., 1996, Proc. Natl. Acad. Sci. 93:3188-3192) or also increased kinetics of clearance. The bacteriophage that are not effectively cleared by the RES, for example in immune compromised patients, or alternatively bacteriophage that intrinsically avoid clearance by the RES, can be passively removed by the therapeutic treatment of the vertebrate with phage specific antiserum.

In another specific embodiment, the present invention contemplates the use of replicating particles, (e.g., alphaviruses), bound to a pathogen-specific moiety, that are themselves minimally virulent to the vertebrate being treated.

In a specific embodiment, the pathogen-specific particle of the invention is a bacteriophage expressing, as a fusion protein, an antibody or fragment or derivative thereof to a pathogen such as an infectious disease agent.

In a preferred embodiment, the particles of the invention are cleared by the reticuloendothelial system RES. In an alternative embodiment, the particles exhibit vascular clearance by a mechanism other than by the RES. In a specific embodiment, suitable particles are cleared from the circulation of the host animal by 90% in 48 hours, more preferably 36 hours, and most preferably 24 hours.

In a preferred embodiment, the particles are of uniform size and charge which is a property termed “monodispersity”. (Reichard and Filkins, 1984, The Reticuloendothelial System; A Comprehensive Treatise, pp. 73-101 (Plenum Press)). Monodispersity is important because it prevents differing clearance rates within the same group of particles, and thus prevents particles that are not effectively cleared from creating a complex with the pathogen targeted for clearance, which then blocks the binding of the pathogen with a particle that will be effectively cleared from the bloodstream or surrounding tissues.

In a preferred embodiment, the particles are of a size range that is 0.01 to 10 microns. In another specific embodiment, the size range of the particles is 0.01 to 1 microns, or 0.1 to 1 microns.

The particles of the present invention are preferably stable in the serum of the host animal to which they are administered, i.e., they are not destroyed and do not disintegrate prior to vascular clearance by the reticuloendothelial system (RES); however, once the particles are endocytosed by phagocytes of the RES, it is preferable that the particles are destroyed once endocytosed by the phagocyte and are not toxic to the phagocytes. Furthermore, the particles preferably do not clump or embolize in the circulation.

### 4.1.1 BACTERIOPHAGE FOR USE IN THE INVENTION

In a preferred embodiment, bacteriophage are utilized as the particle of the invention that is cleared efficiently from the circulation. Bacteriophage have several advantages that make them well suited for use in the present invention including the ability to be manipulated genetically to suit a therapeutic treatment as well as all of the desired properties of clearance of a particle described in Section 4.1 (i.e., monodispersity, clearance by the RES, and selection in vivo and in vitro for increased kinetics of clearance.)

Prior experiments have focused on the use of bacteriophage as an antibacterial agent against the bacteriophage's natural target bacteria. These experiments have been hampered by the fact that bacteriophage are rapidly cleared from the circulation, primarily by the reticuloendothelial system (Merril et al., 1996, Proc. Nat. Acad. Sci. 93:3188-3192; Inchley, 1969, Clin. Exp. Immunol. 5:173-187). In contrast, the present invention makes preferable use of the rapidly cleared bacteriophage.

In a preferred embodiment, the bacteriophage of the present invention are selected for rapid clearance from the circulation in vitro. One can correlate the rate of clearance from the circulation with the column elution profile in vitro. By way of example but not limitation, the skilled artisan collects fractions of bacteriophage from a hydroxylapatite column, and assays the rate of clearance from the circulation for each fraction in vivo in an art accepted test animal. In this manner, an in vitro chromatographic elution profile can be established for particular bacteriophage to select for desirable kinetics of clearance from the circulation.

In a particular example, viral elution profiles on hydroxylapatite columns have been shown to indicate rapid or delayed clearance rates in vivo. This has been done with Alphaviruses and Venezuelan Encephalitis Viruses. Hydroxylapatite column eluted fractions of the viruses were shown to have altered infectivity and clearance rates in vivo compared to wild type. See Jahrling and Beall, 1977, J. Clin. Micro. 6:238-243; Jahrling and Eddy, 1977, Am. J. Epid. 106:408.

Based on the extensive sequence information available for various strains of bacteriophage structural proteins, predictive molecular modifications may also be incorporated to obtain enhanced vascular clearance of the bacteriophage. For example, it has been described that the charges of particular amino acids in a bacteriophage structural protein, correlate with rate of clearance from the circulation (Merril et al., 1996, Proc. Natl. Acad. Sci. 93:3188-3192). In particular, positively charged amino acid residues at a specific position in the structural protein may serve to delay bacteriophage clearance from the circulation and negatively or neutrally charged residues at the same or similar sites may enhance the rate of clearance.

In an alternative embodiment, the bacteriophage are selected in vivo for efficient clearance from the vertebrate circulation prior to their use as a therapeutic. Merril et al., have shown that bacteriophage can be selected in vivo for mutant strains that are able to evade the host's defense mechanisms and hence are more slowly cleared from the circulation (Merril et al., 1996, Proc. Natl. Acad. Sci. 93:3188-3192). In contrast, for use in the present invention, in a particular embodiment, bacteriophage or particles are selected in vivo that are rapidly cleared from the circulation as indicated by, for example, 90% clearance in 48 hours, or more preferably 90% clearance in 36 hours, or most preferably within 90% clearance in 24 hours.

In an alternative embodiment, passive immunization of a bacteriophage that is not effectively cleared by the RES can be used in the methods and compositions of the present invention. The bacteriophage may be opsonized with antibody specific to the bacteriophage (or particle) prior to therapeutic administration, or concurrently, or after therapeutic administration in vivo (by administration of bacteriophage-specific antibody to the patient) to effect the clearance of the immunotherapeutic complex from the circulation.

In another alternative embodiment, prior to disease treatment, the particle of the invention is injected into the subject to be treated as an immunogen to stimulate an immune response to the particle. Subsequently, particles conjugated to a pathogen binding moiety are used as a therapeutic agent to treat a disease. The therapeutic particle conjugated to an antigen binding moiety is rapidly cleared from the circulation by a specific, preformed immune response.

In a preferred embodiment, thoroughly characterized bacteriophage such as a lambda bacteriophage, or a lambda variant, P22 or a P22 variant, M13, M15, fd, f1, or PhiX174, is used as the cleared particle, bound to a pathogen-specific binding moiety, in the methods of the present invention, however, other variants of bacteriophage are clearly contemplated and are within the scope of the present invention.

### 4.2 PATHOGEN-SPECIFIC BINDING MOIETIES

The recognition elements of the pathogen-specific binding moiety may be based upon virtually any known supramolecular interaction such as, for example, nucleotide binding, enzyme-substrate binding, lectin-carbohydrate interactions, antibody-antigen binding, host-guest complexation, and the like. Thus, the binding moiety can be one member of the foregoing binding pairs; the other member being expressed by the pathogen.

In the absence of a previously defined binding moiety, one of ordinary skill in the art would be able to identify a binding moiety using commonly known techniques, such as phage display libraries, combinatorial chemistry, or antibody generation techniques commonly known in the art. Further alternatives may include using chemical moieties derived from proteins, nucleic acids, lipids or carbohydrates that bind pathogenic or otherwise injurious substances.

Numerous techniques have been developed which can be used to identify moieties that bind to pathogens. These techniques include combinatorial chemistry, or phage display libraries from which peptides or proteins are identified.

Combinatorial chemistry can be used to identify binding moieties. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, H., 1997 Journal of Medicinal Chemistry, 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (in the instant invention, a protein or receptor of a pathogen.) The fingerprints are compared with fingerprints obtained from other compounds known to react with the protein of interest to predict whether the library compound might similarly react. For example, rather than testing every ligand in a large library for interaction with a known receptor that is of a pathogen of interest, only those ligands having a fingerprint similar to other compounds known to have that activity could be tested. (See, e.g., Kauvar, L. M. et al., 1995 Chemistry and Biology 2:107-118; Kauvar. L. M., 1995, Affinity fingerprinting, Pharmaceutical Manufacturing International. 8:25-28; and Kauvar, L. M., Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312). The binding moiety need only contact the pathogen to be cleared from the circulation, i.e. the methods and compositions of the present invention do not require the identification of a biological function, other than binding ability, for the binding moiety.

Phage display can be used to screen for peptides and antibodies that bind to a target (e.g., antigen of a pathogen). Various different bacteriophage and methods of expressing the test peptides and antibodies have been reported and can be used. For example, M13 has been extensively characterized and it is known that the viral capsid protein of M13, protein III (pIII), is responsible for infection of bacteria. Several investigators have determined from mutational analysis that the 406 amino acid long pIII capsid protein has two domains. The C-terminus anchors the protein to the viral coat, while portions of the N-terminus of pIII are essential for interaction with the E. coli pilin protein (Crissman, J. W. and Smith, G. P., 1984, Virology 132:445-455). Although the N-terminus of the pIII protein has been shown to be necessary for viral infection, the extreme N-terminus of the mature protein does tolerate alterations. In 1985, George Smith published experiments reporting the use of the pIII protein of bacteriophage M13 as an experimental system for expressing a heterologous protein on the viral coat surface (Smith, G. P., 1985, Science 228:1315-1317). It was later recognized, independently by two groups, that the M13 phage pIII gene display system could be a useful one for mapping antibody epitopes (De la Cruz, V., et al., 1988, J. Biol. Chem. 263:4318-4322; Parmley, S. F. and Smith, G. P., 1988, Gene 73:305-318).

Parmley, S. F. and Smith, G. P., 1989, Adv. Exp. Med. Biol. 251:215-218 suggested that short, synthetic DNA segments cloned into the pIII gene might represent a library of epitopes. These authors reasoned that since linear epitopes were often approximately 6 amino acids in length, it should be possible to use a random recombinant DNA library to express all possible hexapeptides to isolate epitopes that bind to antibodies. Scott, J. K. and Smith, G. P. (1990, Science 249:386-390) describe construction and expression of an “epitope library” of hexapeptides on the surface of M13. Cwirla, S. E., et al., 1990, Proc. Natl. Acad. Sci. USA 87:6378-6382 also described a somewhat similar library of hexapeptides expressed as gene pIII fusions of M13 fd phage. PCT Application WO 91/19818 published Dec. 26, 1991 by Dower and Cwirla describes a similar library of pentameric to octameric random amino acid sequences. Devlin et al., 1990, Science, 249:404-406, describes a peptide library of about 15 residues generated using an (NNS) coding scheme for oligonucleotide synthesis in which S is G or C. Christian and colleagues have described a phage display library, expressing decapeptides (Christian, R. B., et al., 1992, J. Mol. Biol. 227:711-718).

Other investigators have used other viral capsid proteins for expression of non-viral DNA on the surface of phage particles. For example, the major capsid protein pVIII was so used by Cesareni, G., 1992, FEBS Lett. 307: 66-70.

Other bacteriophage than M13 have been used to construct peptide libraries. Four and six amino acid sequences corresponding to different segments of the Plasmodium falciparum major surface antigen have been cloned and expressed in the filamentous bacteriophage fd (Greenwood, J., et al., 1991, J. Mol. Biol. 220:821-827).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about amino acids in length. Such libraries can be advantageously screened to identify peptides, polypeptides and/or other proteins having binding specificity for a variety of ligands. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

McCafferty et al., 1990, Nature 348:552-554 used PCR to amplify immunoglobulin variable (V) region genes and cloned those genes into phage expression vectors. The authors suggested that phage libraries of V, diversity (D), and joining (J) regions could be screened with antigen. The phage that bound to antigen could then be mutated in the antigen-binding loops of the antibody genes and rescreened. The process could be repeated several times, ultimately giving rise to phage which bind the antigen strongly.

Marks et al., 1991, J. Mol. Biol. 222:581-597 also used PCR to amplify immunoglobulin variable (V) region genes and cloned those genes into phage expression vectors.

Kang et al., 1991, Proc. Natl. Acad. Sci. USA 88:4363-4366 created a phagemid vector that could be used to express the V and constant (C) regions of the heavy and light chains of an antibody specific for an antigen. The heavy and light chain V-C regions were engineered to combine in the periplasm to produce an antibody-like molecule with a functional antigen binding site. Infection of cells harboring this phagemid with helper phage resulted in the incorporation of the antibody-like molecule on the surface of phage that carried the phagemid DNA. This allowed for identification and enrichment of these phage by screening with the antigen. It was suggested that the enriched phage could be subject to mutation and further rounds of screening, leading to the isolation of antibody-like molecules that were capable of even stronger binding to the antigen.

Hoogenboom et al., 1991, Nucleic Acids Res. 19:4133-4137 suggested that naive antibody genes might be cloned into phage display libraries. This would be followed by random mutation of the cloned antibody genes to generate high affinity variants.

### 4.3 ANTIBODIES AND ANTIBODY FRAGMENTS

A preferred embodiment of the present invention is the use of an antibody or antibody fragment as the pathogen binding moiety. In particular, the present invention contemplates the use of an isolated Fab single chain or any other antibody fragment that mediates binding to the pathogen that is targeted for clearance from the circulation. The protein/antibody isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.).

According to the invention, the pathogenic agent involved in the disease or disorder to be treated, its antigens, or fragments or other derivatives thereof, or analogs thereof, may be used as an immunogen to generate antibodies which immunospecifically bind such an immunogen. Such antibodies, or derivatives thereof, can be used as the binding moiety in the pathogen-specific particles of the invention. Such antibodies include but are not limited to polyclonal, monoclonal, chimeric, single chain (scFv), Fab fragments, and an Fab expression library. In a specific embodiment, fragments of an pathogen protein identified as hydrophilic are used as immunogens for antibody production.

Various procedures known in the art may be used for the production of polyclonal antibodies to a pathogen or derivative or analog. In a particular embodiment, rabbit polyclonal antibodies to an epitope of a pathogen, or a subsequence thereof, can be obtained. For the production of antibodies, various host animals can be immunized by injection with the native pathogen protein, or a synthetic version, or derivative (e.g., fragment) thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed to a pathogen sequence or analog thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (see e.g., PCT/US90/02545).

According to the invention, human antibodies may be used and can be obtained by using human hybridomas (Cole et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96).

Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for a pathogen together with genes from a human antibody molecule of appropriate biological activity can be used; such antibodies are within the scope of this invention.

Moreover, complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Reichmann et al. (1988, Nature, 332:323; antibodies against hepatitis B in Cole et al. (1991, Proc. Natl. Acad. Sci. USA 88:2869); as well as against viral antigens-respiratory syncytial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling. See Queen, U.S. Pat. No. 5,585,089.

In other embodiments of the invention, techniques described for the production of single chain antibodies (scFv; U.S. Pat. No. 4,946,778) can be adapted to produce pathogen-specific single chain antibodies. An additional embodiment of the invention utilizes the techniques described for the construction of Fab′ expression libraries (Huse et al., 1989, Science 246:1275-1281) to allow rapid and easy identification of monoclonal Fab fragments with the desired specificity for pathogen proteins, derivatives, or analogs.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished by techniques known in the art (e.g., enzyme-linked immunosorbent assay or ELISA). For example, to select antibodies which recognize a specific domain of a pathogen protein, one maylassay generated hybridomas for a product which binds to a fragment containing such domain.

For selection of an antibody that specifically binds a first pathogen but which does not specifically bind a different pathogen, one can select on the basis of positive binding to the first pathogen and a lack of binding to the second pathogen.

### 4.4 CHEMICAL CROSSLINKING A PARTICLE AND A PATHOGEN-SPECIFIC BINDING MOIETY

In one embodiment of the invention, the pathogen-specific binding moiety is cross-linked to the particle. Thus, in a particular embodiment, the binding moiety is bound to the particle other than via a peptide bond.

The chemistry of cross-linking is well known in the art. The nature of the crosslinking reagent used to conjugate the pathogen-specific binding moiety, (i.e., whole IgGs or antibody fragments) and the particle to be delivered can be any suitable reagent known in the art. Any crosslinking agent may be used provided that: a) the clearance of the particle by the RES is retained; and b) the binding to the pathogen by the binding moiety of the therapeutic conjugate is not adversely affected.

The particle may be a fragment of a bacteriophage or bacteria as it is not necessary for the particle to be viable.

In a preferred embodiment, the pathogen-specific binding moiety crosslinked to the particle is an antibody.

In an alternative embodiment, more than one pathogen-specific binding moiety is crosslinked to the particle, thereby creating a particle that can bind to more than one pathogenic agent.

Cross-linking reagents that can be used include but are not limited to p-Azidobenzoyl hydrazide, N-(4-[p-Azidosalicyclamido]-butyl)-3′(2′-pyridyldithio)-propionamide, Bis(beta-[4-azidosalicylamido]-ethyl)disulfide, 1,4-bismaleimidyl-2,3-dihydroxybutane, 1,6-Bismaleimidohexane, 1,5-Difluoro-2,4-dinitrobenzene, Dimethyl adipimidate-2HCl, Dimethyl suberimidate-2HCl, Dimethyladipodimidate-2HCl, Dimethyl pimelimidate-2HCl, Disuccinimidyl glutarate, Disuccinimidyl tartrate, 1-Ethyl-3-[3-Dimethylanonopropyl]Carbodiimide Hydrochloride, (N-Hydroxy succinimidyl)-4-Azidosalicylic acid, Sulfosuccinimdyl 2-[7-azido-4-methyl-coumarin-3-acetamidomethyl-1,3-aminopropionate, N-Succinimidyl-4-iodoacetylaminobenzoate, N-Succinimidyl-3-[2-pyridylthio]propionate, and Succinimidyl 6-[3-(2-pyridylathio)-propionamide]hexanoate (Pierce Chemical Co., Rockford, Ill.).

A specific example of one-step crosslinking of a bacteriophage particle and a binding moiety is oxidation of the bacteriophage with sodium periodate in sodium phosphate buffer for 30 minutes at room temperature, followed by overnight incubation at 4° C. with the compound to be conjugated.

Conjugation also may be performed by derivatizing both the binding moiety and bacteriophage with suffosuccinimidyl 6-[3-(2-pyridyldithio) propionamidel hexanoate (sulfo-LC-SPDP, Pierce Chemical Co., Rockford, Ill.) for 18 hours at room temperature. Therapeutic conjugates also may be prepared by derivatizing binding moieties and the desired compound to be delivered with different crosslinking reagents that will subsequently form a covalent linkage. An example of this reaction is derivatization of bacteriophage with sulfosuccinimidyl 4-(N-maleimidomethyl) cyclo-hexane-1-carboxylate (Sulfo-SMCC, Pierce Chemical Co., Rockford, Ill.), wherein the binding moiety to be conjugated to the bacteriophage is thiolated with N-succinimidyl S-acetylthioacetate (SATA).

In a specific embodiment, the derivatized components are purified free of crosslinker and combined at room temperature for one hour to allow crosslinking. Other crosslinking reagents comprising aldehyde, imide, cyano, halogen, carboxyl, activated carboxyl, anhydride and maleimide functional groups are known to persons of ordinary skill in the art and also may be used for conjugation of compounds to a bacteriophage. The choice of cross-linking reagent will, of course, depend on the nature of the compound desired to be conjugated to the bacteriophage. The crosslinking reagents described above are effective for protein-protein conjugations. If the compound to be conjugated is a carbohydrate or has a carbohydrate moiety, then heterobifunctional crosslinking reagents such as ABH, M2C2H, MPBH and PDPH are useful for conjugation with a proteinaceous pathogen-specific binding moiety (Pierce Chemical Co., Rockford, Ill.).

Another method of conjugating proteins and carbohydrates is disclosed by Brumeanu et al. (Genetic Engineering News, Oct. 1, 1995, p. 16). If the binding moiety to be conjugated is a lipid or has a lipid moiety which is convenient as a site of conjugation for the binding moiety, then crosslinkers such as SPDP, SMPB and derivatives thereof may be used (Pierce Chemical Co., Rockford, Ill.).

It is also possible to conjugate any molecule which is to be delivered by noncovalent means. One convenient way for achieving noncovalent conjugation is to raise antibodies to the binding moiety to be delivered, such as monoclonal antibodies, by methods well known in the art, followed by covalent attachment of said antibody to the particle. The binding moiety is then bound to the monoclonal antibody carrier.

In all of the above crosslinking reactions it is important to purify the derivatized compounds free of crosslinking reagent. It is important also to purify the final conjugate substantially free of unconjugated reactants. Purification may be achieved by affinity, gel filtration or ion exchange chromatography based on the properties of either component of the conjugate. A particularly preferred method is an initial affinity purification step using protein-A Sepharose to retain free, uncrosslinked antibody, followed by gel filtration or ion exchange chromatography based on the mass, size or charge of the particle and binding moiety conjugate.

### 4.5 CONSTRUCTION OF ANTI-PATHOGEN BACTERIOPHAGE

Any of many phage display libraries known in the art can be used to isolate bacteriophage which are bound to a pathogen-specific moiety (as a fusion protein of the bacteriophage), by screening the phage display libraries with the pathogen of interest or a protein or fragment thereof, and identifying bacteriophage that specifically bind to a specific protein/peptide.

In a less preferred embodiment, the protein/peptide portion of a fusion protein identified in phage display library screening, which is able to bind to the pathogen, can be synthesized and covalently bound to the desired bacteriophage by chemical cross-linking (see Section 4.4) or any other methods known in the art.

In a less preferred embodiment, high affinity binding interactions, such as biotin/avidin, can be utilized to non-covalently adhere the particle to a pathogen-specific binding moiety.

Many suitable biological peptide libraries and combinatorial chemistry libraries are known in the art and can be used in the methods of the present invention to isolate a phage which is expressing the pathogen-specific binding moiety as a fusion protein.

Peptides have been expressed in biological systems as fusion proteins with viral structural proteins so as to be expressed on the bacteriophage's outer surface. In a preferred embodiment, a recombinant bacteriophage is used as a therapeutic of the present invention. Bacteriophage are advantageous because they are efficiently cleared by the reticuloendothelial system. Furthermore, many bacteriophage partial or full length genomes are sequenced which makes recombinant expression of foreign gene sequences simpler.

Moreover, the structural proteins of many bacteriophage are well defined which facilitates the task of cloning or crosslinking a binding moiety into or onto a bacteriophage so that the binding moiety is expressed on the surface of the bacteriophage allowing for proximal contact with the targeted pathogen.

In an alternative embodiment, the bacteriophage is crosslinked to the pathogen binding moiety (see Sections 4.4 and 5.4).

The biological peptide libraries discussed above are meant to be illustrative and not limiting. It will be recognized by one of ordinary skill in the art that many other biological peptide libraries may be suitable for use in the practice of the present invention, including but not limited to those described in Section 4.2.

### 4.5.1 ANTIBODY CLONING INTO PHAGE

In a specific embodiment, the pathogen-specific particle of the invention is a bacteriophage that recombinantly expresses on its surface the antigen-binding portion of an antibody to a pathogen.

In a preferred embodiment, a phage display library is screened to identify a binding moiety in the same type of bacteriophage that is to be used for therapeutic treatment, thereby obviating the need to subclone and accelerating the process of preparing a therapeutic agent.

Using phage display libraries, up to 10¹² different antibodies have been expressed on the surface of fd filamentous phage, creating a “single pot” in vitro immune system of antibodies (Griffiths et al., 1994, EMBO J. 13:3245-3260). Selection of desired binding domains from the pool of antibodies in these libraries can be done by techniques known in the art, including contacting the phage to immobilized target protein (i.e. panning), selecting the phage bound to the target. Alternatively, the phage bound to the target can be isolated and the antibody variable regions sub-cloned into an appropriate vector or bacteriophage expressing a desired antibody format (Chapter 17, 1997, Current Protocols in Immunology, John Wiley & Sons, Inc.).

In another embodiment, a gene encoding an antibody that is the pathogen-specific binding moiety of the invention is cloned into a bacteriophage so as to express the antibody as a fusion protein on the phage surface. For example, a pathogen binding moiety portion of an antibody fused to the lambda phage D protein can be cloned in frame to the antibody gene using cre/lox cloning and selection technique (Sternberg and Hoess, 1995, Proc. Natl. Acad. Sci. 92:1609-1613).

Expressing a pathogen-specific binding moiety as a fusion protein on the surface of a bacteriophage results in a therapeutic agent with multiple copies of the binding moiety on its surface. For example, the D protein of lambda phage has 405 copies on the surface of the phage. Cloning a pathogen-specific binding moiety as a fusion protein of the lambda D protein would create a multivalent therapeutic that would be capable of binding multiple pathogens simultaneously to create an immune complex that will be even more efficiently cleared from the circulation by the RES than the bacteriophage alone.

### 4.6 TREATMENT OF DISEASES

The present invention provides methods of treating or preventing a disease or disorder associated with the presence of a pathogen. The pathogen can be any substance that is present in the circulation or is potentially injurious to the host animal, including but not limited to infectious agents, toxins, immune complexes, or drugs. In a preferred embodiment, the pathogen is an infections agent. As discussed in detail below, the infectious agents include, but are not limited to, viruses, bacteria, fungi, protozoa, and parasites. In an alternative embodiment, the pathogen-specific binding moiety targets an antigen which is associated with the proliferation of the infectious agent.

In one embodiment of the invention, the pathogenic agents sought to be cleared from the circulation by the methods and compositions of the present invention encompass any serum drug, including but not limited to barbiturates, tricyclic antidepressants, and Digitalis.

In an alternative embodiment, the pathogenic agents sought to be cleared from the circulation includes naturally occurring substances. Examples of naturally occurring pathogenic agents that could be removed by the methods and compositions of the present invention include but are not limited to low density lipoproteins, interleukins or other immune modulating chemicals and hormones.

In an alternative embodiment, the pathogen sought to be cleared from the circulation includes autoimmune causing factors. These factors include but are not limited to auto-antibodies or naturally occurring molecules associated with autoimmune diseases.

The subject to which a pathogen-specific particle of the invention is administered for therapeutic or prophylactic purposes is any vertebrate, including but not limited to non-human animals, birds, or mammals such as domestic animals (e.g., horses, cows, pigs, dogs, cats, sheep, goats, mice, rats, etc.), and in a preferred embodiment is a primate or human.

Preferred characteristics of a vertebrate treated with the methods and compositions of the present invention include blood flow to liver and the presence of fixed tissue macrophages (Kupffer cells).

Moreover, the availability of serum opsonins that bind to the particle that are either already present in the vertebrate's serum or can be added to subsequently opsonize the particle is important.

In a specific embodiment, the pathogen-specific particle of the invention that is administered is purified.

In a preferred embodiment, the therapeutic agent of the invention specifically binds an antigen of the pathogenic agent of an infectious disease. The pathogen-specific binding moiety is preferably an antibody. In an alternative embodiment, the pathogen-specific binding moiety is a cellular receptor for the infectious disease agent, or a fragment of the host cellular receptor to which the pathogenic agent binds.

In a specific embodiment, the pathogen-specific binding moiety can be any substance that binds the pathogenic agent involved in the disease or disorder sought to be treated, for example, a cellular receptor of the pathogenic agent. Cellular receptors that can be utilized as pathogen-specific binding moieties for treatment of an infectious disease are listed in Table 1, along with the pathogenic agent which binds to the cellular receptor. Fragments or derivatives of such cellular receptors that retain binding specificity can also be used.

TABLE 1

| Pathogen | Cellular Receptor |
|---|---|
| B-lymphotropic | LPV receptor on B-cells |
| papovavirus (LPV) | |
| Bordatella pertussis | Adenylate cyclase |
| Borna Disease virus | BDV surface glycoprotiens |
| (BDV) | |
| Bovine coronavirus | N-acetyl-9-O-acetylneuraminic acid |
| | receptor |
| Choriomeningitis virus | CD4+ |
| Dengue virus | Highly sulphated type Heparin |
| | sulphate |
| | p65 |
| E. coli | Gal alpha 1-4Gal-containing |
| | isoreceptors |
| Ebola | CD16b |
| Echovirus 1 | Integrin VLA-2 receptor |
| Echovirus-11 (EV) | EV receptor |
| Endotoxin (LPS) | CD14 |
| Enteric bacteria | Glycoconjugate receptors |
| Enteric Orphan virus | alpha/beta T-cell receptor |
| Enteroviruses | Decay-accelerating factor receptor |
| Feline leukemia virus | Extracellular envelope glycoprotein |
| | (Env-SU) receptor |
| Foot and mouth disease | Immunoglobulin Fc |
| virus | receptorPoxvirusM-T7 |
| Gibbon ape leukemia | GALV receptor |
| virus (GALV) | |
| Gram-negative bacteria | CD14 receptor |
| Heliobacter pylori | Lewis(b) blood group antigen |
| | receptor |
| Hepatitis B virus (HBV) | T-cell receptor |
| Herpes Simplex Virus | Heparin sulphate glycoaminoglycan |
| | receptor |
| | Fibroblast growth factor receptor |
| HIV-1 | CC-Chemokine receptor CCR5 |
| | CD11a |
| | CD2 |
| | G-protein coupled receptor |
| | CD4 |
| Human cytomegalovirus | Heparin sulphate proteoglycan |
| | Annexin II |
| | CD13 (aminopeptidase N) |
| Human coronovirus | Human aminopeptidase N receptor |
| Influenza A, B & C | Hemagglutinin receptor |
| Legionella | CR3 receptor |
| | Protein kinase receptor |
| | Galactose N-acetylgalactosamine |
| | (Gal/GalNAc)-inhibitable lectin |
| | receptor |
| | Chemokine receptor |
| Leishmania mexicana | Annexin I |
| Listeria monocytogenes | ActA protein |
| Measles virus | CD46 receptor |
| Meningococcus | Meningococcal virulence associated |
| | Opa receptors |
| Morbilliviruses | CD46 receptor |
| Mouse hepatitis virus | Carcinoembryonic antigen family |
| | receptors |
| | Carcinoembryonic antigen family |
| | Bgla receptor |
| Murine leukemia virus | Envelope glycoproteins |
| Murine gamma herpes | gamma interferon receptor |
| virus | |
| Murine retrovirus | Glycoprotein gp70 |
| | Rmc-1 receptor |
| Murine coronavirus | Carcinoembryonic antigen family |
| mouse hepatitis virus | receptors |
| Mycobacterium avium-M | Human Integrin receptor alpha v |
| | beta 3 |
| Neisseria gonorrhoeae | Heparin sulphate proteoglycan |
| | receptor |
| | CD66 receptor |
| | Integrin receptor |
| | Membrane cofactor protein |
| | CD46 |
| | GM1 |
| | GM2 |
| | GM3 |
| | CD3 |
| | Ceramide |
| Newcastle disease virus | Hemagglutinin-neuraminidase protein |
| | Fusion protein |
| Parvovirus B19 | Erythrocyte P antigen receptors |
| Plasmodium falciparum | CD36 receptor |
| | Glycophorin A receptor |
| Pox Virus | Interferon gamma receptor |
| Pseudomonas | KDEL receptor |
| Rotavirus | Mucosal homing alpha4beta7 receptor |
| Samonella typhiurium | Epidermal growth factor receptor |
| Shigella | alpha5betal integrin protein |
| Streptococci | Nonglycosylated J774 receptor |
| T-helper cells type 1 | Chemokine receptors including: |
| | 6.CXCR1-4 |
| | 7.CCR1-5 |
| | 8.CXCR3 |
| | 9.CCR5 |
| T-cell lymphotropic | gp46 surface glycoprotein |
| virus 1 | |
| Vaccinia virus | TNFRp55 receptor |
| | TNFRp75 receptor |
| | Soluble Interleukin-1 beta |
| | receptor |

### 4.6.1 INFECTIOUS DISEASES

In specific embodiments, infectious diseases are treated or prevented by administration of a particle bound to a pathogen-specific binding moiety that recognizes an antigen of an infectious disease agent. Such antigen can be but is not limited to: influenza virus hemagglutinin (Genbank accession no. JO2132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), core protein, matrix protein or other protein of Dengue virus (Genbank accession no. M19197; Hahn et al., 1988, Virology 162:167-180), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I) a glycoprotein of La Crosse virus (Gonzales-Scarano et al., 1982, Virology 120:42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), antigen of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

Additional diseases or disorders that can be treated or prevented by the use of particles bound to pathogen-specific binding moieties that recognize the virus causing the disease include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picornaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus 1 (B virus), and poxviruses

Bacterial diseases or disorders that can be treated or prevented by the use of particles bound to pathogen-specific binding moieties that recognize the bacteria causing the disease include, but are not limited to, Mycobacteria rickettsia, Mycoplasma, Neisseria spp. (e.g., Neisseria menigitidis and Neisseria gonorrhoeae), Legionella, Vibrio cholerae, Streptococci, such as Streptococcus pneumoniae, Corynebacteria diphtheriae, Clostridium tetani, Bordetella pertussis, Haemophilus spp. (e.g., influenzae), Chlamydia spp., enterotoxigenic Escherichia coli, etc.

Protozoal diseases or disorders that can be treated or prevented by the use of particles bound to pathogen-specific binding moieties that recognize the protozoans causing the disease include, but are not limited to, plasmodia, eimeria, Leishmania, and trypanosoma.

### 4.7 PHARMACEUTICAL FORMULATION AND ADMINISTRATION

Therapeutic compositions containing a therapeutic agent (i.e., a particle bound to a pathogen-specific moiety) for use in accordance with the present invention can be formulated and administered in any conventional manner using one or more physiologically acceptable carriers or excipients.

Many methods may be used to introduce the therapeutic formulations of the invention; these include but are not limited to intramuscular, intravenous, and subcutaneous (scratching through the top layers of skin, e.g., using a bifurcated needle) or any other standard routes of immunization. In a preferred embodiment, the route of administration is intravenous.

Thus, the therapeutic agents and physiologically acceptable salts and solvents can be formulated for administration by insufflation (either through the mouth or the nose) parenteral or rectal administration.

For administration by inhalation, the therapeutics according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The therapeutics can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. The therapeutics can also be formulated in rectal compositions such as suppositories or retention enemas, e.g., containing conventional suppository bases such as cocoa butter or other glycerides.

In addition to the formulations described previously, the therapeutics can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Therapeutic agents of the invention may be administered as separate compositions or as a single composition with more than one pathogen-specific particle. Additionally, the diagnostic and therapeutic value of the therapeutic agents of the invention may be augmented by their use in combination with other therapeutics for the disease or disorder caused by the pathogenic agent. Additionally, the particle bound to an pathogen-specific binding moiety can be administered in conjunction with an appropriate antibiotic, antifungal, anti-viral or any other drug useful in treating or preventing the infectious disease.

In an alternative embodiment, the particle bound to an pathogen-specific binding moiety is further conjugated to a compound effective against the infectious disease agent to which the particle bound to a pathoget-specific binding moiety is directed, for example, an antibiotic, antifungal or anti-viral agent.

The composition, if desired, can also contain minor amounts of wetting or emulsifying agents, or pH buffering agents. The composition can be a liquid solution, suspension, emulsion, capsule, sustained release formulation, or powder.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Composition comprising a compound of the invention formulated in a compatible pharmaceutical carrier may also be prepared, placed in an appropriate container, and labeled for treatment of an indicated condition.

Suitable preparations of the therapeutics of the invention include injectables, either as liquid solutions or suspensions; solid forms suitable for solution in, suspension in, liquid prior to injection, may also be prepared. The preparation may also be emulsified, or the therapeutic particles bound to the pathogen-specific binding moiety can be encapsulated in liposomes. The active immuno-therapeutic ingredients are often mixed with excipients which are pharmaceutically acceptable and compatible with the active ingredient. Suitable excipients are, for example, water, saline, buttered saline, dextrose, glycerol, ethanol, sterile isotonic aqueous buffer or the like and combinations thereof. In addition, if desired, the therapeutics of the invention may also include minor amounts of auxiliary substances such as wetting or emulsifying agents, and/or pH buffering agents.

Examples of adjuvants which may be effective, include, but are not limited to: aluminum hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

In a specific embodiment, a lyophilized particle bound to a pathogen-specific binding moiety of the invention is provided in a first container; a second container comprises diluent consisting of an aqueous solution of 50% glycerin, 0.25% phenol, and an antiseptic (e.g., 0.005% brilliant green).

### 4.8 EFFECTIVE DOSE OF THE PATHOGEN-SPECIFIC BINDING MOIETY

The precise dose of the particle bound to a pathogen-specific binding moiety to be employed in the formulation will also depend on the route of administration, and the nature of the patient, and should be decided according to the judgment of the practitioner and each patient's circumstances according to standard clinical techniques. Effective doses may also be extrapolated from dose-response curves derived from art accepted animal model test systems.

The particles of the invention described herein can be administered to a patient at a therapeutically effective dose to treat a disease or disorder caused by a pathogen. A therapeutically effective dose refers to that amount sufficient to result in at least a minimally healthful benefit in the treated subject.

In a preferred embodiment, the dose of the pathogen-specific particle is based upon the concentration of the pathogenic agent in the circulation (i.e., the blood). The concentration of the pathogenic agent in the circulation can be estimated by any commercially available or standardized hematological test for the particular pathogenic agent to be treated, or any other method known in the art. The pathogen-specific particle is then formulated, as described in Section 4.7, at a concentration range that is preferably at least ten fold in excess of the subject's systemic concentration of the pathogenic agent and administered intravenously. More preferably, the pathogenic-specific binding moiety is administered at a concentration that is more than one hundred fold more than the concentration of the pathogenic agent.

For example, during an infection 10⁴-10⁹ pathogens per millimeter are circulating, therefore, the number of pathogen-specific particles administered to the subject is preferably ten fold greater, that is at least 10⁵-10¹⁰ pathogen-specific particles per millimeter. Determination of the number of pathogen-specific particles for use in a dose will be derived empirically based upon the particle and pathogen-specific binding moiety used and the concentration of the pathogen to be treated.

Toxicity and therapeutic efficacy of particles can be determined by standard pharmaceutical procedures in cell cultures or experimental animals, e.g. , for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Particles which exhibit large therapeutic indices are preferred. While particles that exhibit toxic side effects can be used, care should be taken to design a delivery system that targets such particles to the site of affected tissue in order to minimize potential damage to uninfected cells and, thereby, reduce side effects.

The data obtained from the cell culture assays and animal studies can be used in deriving a range of dosage for use in humans. The dosage of such particles lies preferably within a range of circulating concentrations that include the ED₅₀ with little or no toxicity. The dosage can vary within this range depending upon the dosage form employed and the route of administration utilized.

For any therapeutic particle used in the method of the invention, dose can be formulated in animal models to achieve a circulating plasma concentration range that includes the IC₅₀ (i.e., the concentration of the test particle which achieves a half-maximal inhibition of symptoms). Such information can be used to more accurately determine useful doses in humans.

### 4.9 DEMONSTRATION OF THERAPEUTIC UTILITY

The therapeutics of the invention are preferably tested in vitro, and then in vivo, for the desired therapeutic or prophylactic activity, prior to use in humans.

The therapeutics are preferably tested for efficacy in appropriate animal models, and in clinical trials, in humans. The efficacy of the therapeutic may be determined by any method in the art, for example, after administration of the therapeutic to the animal model or to the human subject, the animal or human subject is evaluated for any indicator of the disease or disorder that the therapeutic is intended to treat. For example, the efficacy of the therapeutic can be assessed by measuring the level of the pathogen in the blood against which the particle bound to the pathogen-specific binding moiety is directed in the animal model or human subject at suitable time intervals before, during, or after therapy. Any change or absence of change in the amount of the pathogen can be identified and correlated with the effect of the treatment on the subject. The level of the pathogen can be determined by any method known in the art.

In other aspects, the particle bound to a pathogen-specific binding moiety may be tested for efficacy by monitoring the subject for improvement or recovery from the particular disease or condition associated with the pathogen against which the particle bound to a pathogen-specific binding moiety is directed. Before carrying out such trials in humans, the tests for efficacy of the therapeutic agents can be performed in animal models of the particular disease or disorder caused by the pathogen.

Where the therapeutic of the invention is specific for an antigen of an infectious disease agent, the therapeutic efficacy of the particle bound to a pathogen-specific binding moiety can be assayed by administering the particle bound to a pathogen-specific binding moiety to a subject (either a human subject or an animal model for the disease) and then monitoring either the levels of the particular infectious disease agent or symptoms of the particular infectious disease. The levels of the infectious disease agent may be determined by any method known in the art, for assaying the levels of an infectious disease agent, e.g., the viral titer, in the case of a virus, or bacterial levels (for example, by culturing of a sample from the patient), etc. The levels of the infectious disease agent may also be determined by measuring the levels of the antigen against which the particle bound to a pathogen-specific binding moiety was directed. A decrease in the levels af the infectious disease agent or an amelioration of the symptoms of the infectious disease indicates that the particle bound to a pathogen-specific binding moiety is effective.

Where the therapeutic of the invention is administered, the potency of the therapeutic formulation containing the particle bound to a pathogen-specific binding moiety of the invention can be determined by monitoring the level of the pathogen in test animals following treatment. Test animals may include but are not limited to mice, rabbits, chimpanzees and eventually human subjects. A therapeutic made in this invention can be made to treat chimpanzees experimentally. However, since chimpanzees are a protected species, the therapeutic response to particle bound by a pathogen-specific binding moiety of the invention can first be studied in a number of smaller, less expensive animals, with the goal of finding one or two best candidate particles bound to a pathogen-specific binding moiety or best combinations of particles bound to a pathogen-specific binding moiety to use in chimpanzee efficacy studies.

The clearance of the pathogen from the circulation of the test subjects can be analyzed by various approaches such as the reactivity of the serum to binding moieties specific to the pathogen, as assayed by known techniques, e.g., enzyme linked immunosorbent assay (ELISA), immunoblots, radio-immunoprecipitations, etc. It is preferable that the monitoring of the clearance from the circulation of the pathogenic agent by the therapeutic of the invention begin within one hour and extend for up to one week or more to ensure that the clearance of the pathogenic agent was complete and there is no recurrence of disease.

The following examples are presented by way of illustration of the previously described invention and are not limiting of that description.

### 4.10 DIAGNOSTIC METHODS

The particles of the invention that bind a specific molecule of a pathogen that is a member of a binding pair (the binding moiety being the other member) may be used in diagnostics and prognostics, as described herein.

In various embodiments, the present invention provides the measurement of a member of the binding pair, and the uses of such measurements in clinical applications. The therapeutic agents in the present invention may be used, for example, in the detection of an antigen in a biological sample whereby patients may be tested for aberrant levels of the molecule to which the particle bound to a pathogen-specific binding moiety binds, and/or for the presence of abnormal forms of such molecules. The term “aberrant levels” is meant to encompass increased or decreased levels relative to that present, or a standard level representing that present, in an analogous sample from a portion of the body or from a subject not having the disorder. The particles bound to a pathogen-specific binding moiety of this invention may also be included as a reagent in a kit for use in a diagnostic or prognostic technique.

In a specific embodiments of the invention, a particle bound to a pathogen-specific binding moiety of the invention that immunospecifically binds to an antigen of an infectious disease agent may be used to diagnose, prognose or screen for an infectious disease associated with the expression of the antigen of the infectious disease agent. In a preferred aspect of the embodiment, the invention provides a method of diagnosing or screening for the presence of an infectious disease agent, characterized by the presence of an antigen of said infectious disease agent, which antigen is a first member of a binding pair consisting of said first member and a second member, said method comprising measuring in a subject the level of immunospecific binding of a particle bound to a pathogen-specific binding moiety to a sample derived from the subject, in which said particle bound to a pathogen-specific binding moiety immunospecifically binds said antigen and in which said particle bound to a pathogen-specific binding moiety is detectable by a standard assay and indicates the presence of said infectious disease agent.

In another preferred embodiment, the invention provides a method for detecting abnormal levels of a particular ligand or receptor in a sample derived from a subject by comparing the immunospecific binding of a particle bound to a pathogen-specific binding moiety that binds the particular ligand or receptor to the sample with the immunospecific binding of the particle bound to a pathogen-specific binding moiety to a sample having normal levels of the ligand or receptor.

The measurement of a molecule that is bound by a particle bound to a pathogen-specific binding moiety can be valuable in detecting and/or staging diseases related to the molecule in a subject, in screening of such diseases in a population, in differential diagnosis of the physiological condition of a subject, and in monitoring the effect of a therapeutic treatment on a subject.

Assays can be designed to detect the pathogens which are bound by the rapidly cleared particle of the invention which is itself bound to a pathogen-specific binding moiety. In specific embodiments, these diagnostic methods may be used to detect abnormalities in the level of gene expression, or abnormalities in the structure and/or temporal, tissue, cellular, or sub-cellular location of the pathogen to be assayed.

The binding activity of a given particle bound to a pathogen-specific binding moiety may be determined according to well known methods. Those skilled in the art will be able to determine operative and optimal assay conditions for each determination by employing routine experimentation.

One of the ways in which a particle bound to a pathogen-specific binding moiety can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller et al., 1978, J. Clin. Pathol. 31:507-520; Butler, 1981, Meth. Enzymol. 73:482-523; Maggio, E. (ed.), 1980, Enzyme Immunoassay, CRC Press, Boca Raton, Fla.,; Ishikawa et al., (eds.), 1981, Enzyme Immunoassay, Kgaku Shoin, Tokyo)). The enzyme which is bound to the particle bound to a pathogen-specific binding moiety will react with an appropriate substrate, preferably a chromogenic substrate, in such a manner as to produce a chemical moiety which can be detected, for example, by spectrophotometric, fluorimetric or by visual means.

It is also possible to label the particle bound to a pathogen-specific binding moiety with a fluorescent or chemiluminescent or bioluminescent compound, etc.

### 5. EXAMPLE

Construction of Anti-Pathogen Bacteriophage

In the following example, a bacteriophage is utilized as the therapeutic particle of the present invention, fused recombinantly to a single chain antibody that is specific for the hepatitis B surface antigen. The single chain antibody specifically binds the hepatitis B surface antigen. One of ordinary skill in the relevant art would recognize that any particle that is efficiently cleared from the circulation according to the present invention will be appropriate for use. Additionally, the following example utilizes the recombinant expression of a bacteriophage surface protein fused to an antibody, however, one of ordinary skill in the relevant art will recognize that any-pathogen-specific binding moiety according to the invention would be suitable.

### 5.1 BACTERIOPHAGE SELECTION

A lambda bacteriophage is selected in vitro for efficient clearance from the vertebrate circulation prior to use as a therapeutic, using methods as described by Jahrling and Beall, 1977, J. Clin. Micro., 6:238-243 (see also Jahrling and Eddy, 1977, Am. J. Epid. 106:408-419), as follows:

The lambda phage is grown on an E. coli bacterial strain that is susceptible to mutations (e.g., CRM2). The lambda phage is then prepared in large scale stocks using standard bacteriophage purification techniques (Sambrook, et al., 1989, Molecular Cloning, A Laboratory Manual, Second Edition, Cold Spring Harbor Press, N.Y.; and Ausubel, et al., 1989-1999, Current Protocols in Molecular Biology, Green Publishing Associates and Wiley Interscience, N.Y.).

Hydroxylapatite that is commercially prepared (Bio-Rad Laboratories Inc, HTP™) is suspended in six volumes of starting buffer. After removal of fine particles, the slurry is diluted to two volumes of starting buffer per gram of hydroxylapatite, poured into columns, and allowed to settle. The packed bed is adjusted to ten centimeters in length, and 45 ml of starting buffer is washed through the column. In preliminary experiments, columns are checked for channeling or skewing with bromocresol purple.

Virus samples, suspended in 0.5 ml of NTE buffer (0.1 M NaCl-0.02 M Tris (hydroxymethyl) aminomethane-hydrochloride, 0.001 M ethylene diamine tetra acetic acid), are mixed with an equal volume of starting buffer and allowed to enter the column bed. One ml of starting buffer is then applied and a gradient maker is attached (Pharmacia, GM-1), producing a linear phosphate gradient. A total of 100 ml of buffer is used for each column run.

Columns are run under gravity and flow rates monitored. Elution buffers are prepared to elute the phage with increasing concentration of NaCl.

The isolated lambda phage fractions are tested in a mouse to identify the particular fraction with rapid clearance from the circulation. Lambda phage stocks collected from the columns are concentrated and injected into the mouse at 10¹⁰ to 10¹² phage per ml. The titer of the bacteriophage in the circulation for each round of selection is monitored by collecting blood samples from the mice and performing standard plaque assays using the blood samples as the source of virus. By plotting the actual numbers of virus, measured in pfu/milliliter of blood or milliliter of liver tissue, present at 30 minute, one hour, and 3 hour time points on a graph, the practitioner is able to monitor the rate of clearance of the isolated strain of bacteriophage from the circulation.

The column isolation of lambda bacteriophage and animal testing is repeated up to ten times to obtain a lambda bacteriophage strain that is optimized for rapid clearance from the host immune system.

By this method, the column fraction corresponding to bacteriophage that have been rapidly cleared from the circulation (i.e., within thirty minutes) are identified and used in Section 5.2 below.

### 5.2 ANTIBODY CLONING INTO PHAGE

The hepatitis B surface antigen is injected into a mouse with a suitable adjuvant in order to induce an immune response. Production of antibodies to the hepatitis B surface antigen are monitored by ELISA. After an immune reaction is clearly induced, the spleen of the mouse is harvested and B cells isolated for fusion with myeloma cells to generate a monoclonal cell line. The hybridoma cell secreting the desired monoclonal antibody is sub-cloned from the population of hybridomas by standard techniques. In particular, a hybridoma producing a high affinity antibody specific for the hepatitis B surface antigen is selected.

Upon identification of the antibody, the antibody variable heavy chain and variable light chains are amplified by PCR using primers specific for the constant regions. The proper amplification of variable heavy and light chain sequences of the PCR products is verified by sequencing.

The products of the PCR are then amplified again using primers, wherein the variable heavy chain is amplified using the same 5′ primer as before; however, the 3′ primer encodes a joining sequence (i.e. (Gly₄Ser)₃). The 5′ primer of the variable light chain encodes an overlapping sequence corresponding to the 3′ primer of the variable heavy chain and the 3′ primer for amplification of the variable light chain gene is the same as in the first round of PCR. After the second PCR reaction, the variable heavy and light chains are ligated together using standard ligation procedures, resulting in a single chain antibody (scFv) fragment with specificity to the hepatitis B surface antigen. The ligation product is sequenced to confirm an accurate PCR amplification and cloning of the joining sequence.

The amplified and fused scFv gene is then cloned into a lambda bacteriophage in frame with the lambda D protein, using the same bacteriophage that is selected for rapid vascular clearance as described in Section 5.1 above, thereby deriving a HBV-specific bacteriophage.

Molecular cloning of the nucleic acids is done by standard techniques (see e.g., Sambrook et al., Molecular Cloning, A Laboratory Manual, Cold Spring Harbor Press, New York (1989)).

### 5.3 THERAPEUTIC DOSE OF ANTI-HBV THERAPEUTICS

The concentration of hepatitis B virus (HBV) in an infected human subject's circulation is estimated by a commercially available or standardized hematological test. The HBV-specific bacteriophage derived as described in Section 5.2 is then formulated, as described in Section 4.7, at a concentration range that is at least ten fold in excess of the subject's systemic concentration of the pathogenic agent and administered intravenously.

During a typical HBV infection, not more than about 10⁸ infectious HBV per milliliter of blood are circulating; therefore, the number of pathogen-specific particles administered to the human subject is preferably at least 10⁹ therapeutic bacteriophage expressing the anti-hepatitis B surface antibody per milliliter of blood of the subject to be treated. A dose of about 10⁹ therapeutic bacteriophage corresponds to roughly 1 microgram in mass, so that a dose of 10⁹ bacteriophage per ml of blood results in a total body dosage of about 10 milligrams, presuming a blood volume of approximately 10 liters. A particle displaying multiple binding sites, e.g., the HBV-specific bacteriophage, may reduce the minimum required dosage for effective therapeutic treatment in relative proportion to the number of binding sites.

### 5.4 CHEMICAL CROSSLINKING

The following example sets forth an exemplary protocol for preparing the therapeutic particles of the present invention whereby the particle is chemically crosslinked to the pathogen binding moiety. The present example utilizes a bacteriophage (see Section 5.1) and an antibody (see Section 5.2), however, one of ordinary skill in the relevant art will recognize that the techniques of this example can be utilized for crosslinking any appropriate chemical group of a particle of the invention with an appropriate chemical group of a pathogen binding moiety of the present invention.

### 5.4.1 CHEMICAL CROSSLINKING OF ANTIBODIES TO PHAGE

A lambda bacteriophage that is rapidly cleared from the circulation is identified as in Section 5.1, above. A hepatitis B surface antigen specific antibody is identified as in Section 5.2 above.

Conjugation of the antibody and lambda bacteriophage is performed by derivatizing both the antibody and bacteriophage with suffosuccinimidyl 6-[3-(2-pyridyldithio) propionamidel hexanoate (sulfo-LC-SPDP, Pierce Chemical Co., Rockford, Ill.) for 18 hours at room temperature.

The derivatized components are purified free of crosslinker and mixed at room temperature for one hour to allow crosslinking. An initial affinity purification step is employed using protein-A Sepharose to retain free, uncrosslinked antibody, followed by gel filtration.

The number of HBV in the circulation is estimated by any commercially available or standardized hematological test for the particular pathogenic agent to be treated. The pathogen-specific binding moiety is then formulated, as described in Section 4.7 above, at a concentration range that is ten fold in excess of the concentration of the pathogenic agent and administered intravenously.

The present invention is not to be limited in scope by the specific embodiments described herein. Indeed, various modifications of the invention in addition to those described herein will become apparent to those skilled in the art from the foregoing description and accompanying drawings. Such modifications are intended to fall within the scope of the appended claims.

Various references are cited herein above, including patent applications, patents, and publications, the disclosures of which are hereby incorporated by reference in their entireties.

What is claimed is:

1. A method of treating a vertebrate having a disease or disorder associated with the presence of a pathogen comprising administering to the vertebrate a therapeutically effective dose of a particle bound to a binding moiety that specifically binds the pathogen, said binding moiety not being endogenous to said particle.

2. The method according to claim 1 wherein the particle is an insoluble particle consisting essentially of silica, metal oxide, or a suspension of colloidal carbon.

3. The method according to claim 1 wherein the particle is a viral capsid lacking viral nucleic acid.

4. The method according to claim 1 wherein the particle is a nonvirulent, replicative viral particle.

5. The method according to claim 1 wherein the particle consists essentially of denatured protein.

6. The method according to claim 1 wherein the particle is a ghost red blood cell.

7. The method according to claim 1 wherein the particle is a bacteriophage.

8. The method according to claim 1 wherein the particle is 90% cleared from the circulation of the vertebrate within 48 hours.

9. The method according to claim 7 wherein the bacteriophage is selected from the group consisting of lambda, lambda variant, P22, P22 variant, M13, M15, fd, f1, and PhiX174.

10. The method according to claim 1 wherein the binding moiety is cross-linked to the particle.

11. The method according to claim 7 wherein the binding moiety is cross-linked to the particle.

12. The method according to claim 1 wherein the binding moiety is a fusion protein of the particle.

13. The method according to claim 7 wherein the binding moiety is expressed as a fusion protein to a capsid molecule of the bacteriophage or to at least a portion of the capsid molecule functional in capsid assembly.

14. The method according to claim 13 wherein the binding moiety is expressed as a fusion protein to gene D protein of lambda, gene III protein of M13, or gene VIII protein of fd.

15. The method according to claim 1 wherein said administering is intravenous.

16. The method according to claim 1 wherein said vertebrate is a human.

17. The method according to claim 1 wherein said vertebrate is a non-human vertebrate.

18. The method according to claim 1 wherein the binding moiety is one member of a binding pair or a binding portion thereof selected from the group consisting of ligand-receptor, enzyme-substrate, and lectin-carbohydrate, and the other member of the binding pair is expressed on the pathogen.

19. The method according to claim 1 wherein the binding moiety is an antibody or derivative thereof containing the binding domain.

20. The method according to claim 7 wherein the binding moiety is an antibody or derivative thereof containing the binding domain.

21. The method according to claim 10 wherein the binding moiety is an antibody or derivative thereof containing the binding domain.

22. The method according to claim 13 wherein the binding moiety is an antibody or derivative thereof containing the binding domain.

23. The method according to claim 1, 7, or 19 wherein the pathogen is an infectious agent.

24. The method according to claim 23 wherein the infectious agent is a virus.

25. The method according to claim 23 wherein the infectious agent is a bacterium.

26. The method according to claim 23 wherein the infectious agent is a fungus.

27. The method according to claim 23 wherein the infectious agent is a protozoan.

28. The method according to claim 23 wherein the infectious agent is a parasite.

29. A purified particle cross-linked to a binding moiety that specifically binds a pathogen.

30. A purified particle bound to a binding moiety that specifically binds a toxin, immune complex, or drug.

31. The particle of claim 29 wherein the pathogen is an infectious agent of a vertebrate.

32. The particle of claim 31 wherein the infectious agent is a virus.

33. The particle of claim 31 wherein the infectious agent is a bacterium.

34. The particle of claim 31 wherein the infectious agent is a fungus.

35. The particle of claim 31 wherein the infectious agent is a protozoan.

36. The particle of claim 31 wherein the infectious agent is a parasite.

37. The particle of claim 29 or 30 wherein the particle is an insoluble particle consisting essentially of silica, metal oxide, or a suspension of colloidal carbon.

38. The particle of claim 29 wherein the particle is a viral capsid lacking viral nucleic acid.

39. The particle of claim 29 wherein the particle is a nonvirulent, replicative viral particle.

40. The particle of claim 29 wherein the particle consists essentially of denatured protein.

41. The particle of claim 29 wherein the particle is a ghost red blood cell.

42. The particle of claim 29 or 30 wherein the particle is a bacteriophage.

43. A purified particle other than a bacteriophage bound to a binding moiety that specifically binds a pathogen.

44. The particle of claim 29, 31, or 43 wherein upon administration of the particle to a vertebrate, the particle is 90% cleared from the circulation of the vertebrate within 48 hours.

45. The particle of claim 42 wherein the bacteriophage is selected from the group consisting of lambda, lambda variant, P22, P22 variant, M13, M15, fd, f1, and PhiX174.

46. The particle of claim 30 wherein the binding moiety is cross-linked to the particle.

47. The particle of claim 43 wherein the binding moiety is cross-linked to the particle.

48. The particle of claim 30 wherein the binding moiety is a fusion protein of the particle.

49. The particle of claim 48, which is a bacteriophage wherein the binding moiety is expressed as a fusion protein to a capsid molecule of the bacteriophage or to at least a portion of the capsid molecule functional in capsid assembly.

50. The particle of claim 49 wherein the binding moiety is expressed as a fusion protein to gene D protein of lambda, gene III protein of M13, or gene VIII protein of fd.

51. The particle of claim 29 wherein the binding moiety is one member of a binding pair or a binding portion thereof selected from the group consisting of ligand-receptor, enzyme-substrate, and lectin-carbohydrate, and the other member of the binding pair is expressed on the pathogen.

52. The particle of claim 42 wherein the binding moiety is an antibody or derivative thereof containing the binding domain.

53. The particle of claim 46 wherein the binding moiety is an antibody or derivative thereof containing the binding domain.

54. The particle of claim 47 wherein the binding moiety is an antibody or derivative thereof containing the binding domain.

55. A composition comprising the purified particle of claim 29, 30, 31, 42, or 43.

56. A pharmaceutical composition comprising a purified particle bound to a binding moiety that specifically binds a pathogen, said binding moiety not being endogenous to said particle, in an amount effective to treat a vertebrate having a disease or disorder associated with the presence of said pathogen; and pharmaceutically acceptable carrier.

57. The pharmaceutical composition of claim 56 wherein the pathogen is an infectious agent of a vertebrate.

58. The pharmaceutical composition of claim 57 wherein the particle is an insoluble particle consisting essentially of silica, metal oxide, or a suspension of colloidal carbon.

59. The pharmaceutical composition of claim 57 wherein the particle is a viral capsid lacking viral nucleic acid.

60. The pharmaceutical composition of claim 57 wherein the particle is a nonvirulent, replicative viral particle.

61. The pharmaceutical composition of claim 57 wherein the particle consists essentially of denatured protein.

62. The pharmaceutical composition of claim 57 wherein the particle is a ghost red blood cell.

63. The pharmaceutical composition of claim 57 wherein the particle is a bacteriophage.

64. The pharmaceutical composition of claim 57 wherein upon administration of the composition to a vertebrate, the particle is 90% cleared from the circulation of the vertebrate within 48 hours.

65. The pharmaceutical composition of claim 63 wherein the bacteriophage is selected from the group consisting of lambda, lambda variant, P22, P22 variant, M13, M15, fd, f1, and PhiX174.

66. The pharmaceutical composition of claim 57 wherein the binding moiety is cross-linked to the particle.

67. The pharmaceutical composition of claim 63 wherein the binding moiety is cross-linked to the particle.

68. The pharmaceutical composition of claim 57 wherein the binding moiety is a fusion protein of the particle.

69. The pharmaceutical composition of claim 63 wherein the binding moiety is expressed as a fusion protein to a capsid molecule of the bacteriophage or to at least a portion of the capsid molecule functional in capsid assembly.

70. The pharmaceutical composition of claim 69 wherein the binding moiety is expressed as a fusion protein to gene D protein of lambda, gene III protein of M13, or gene VIII protein of fd.

71. The pharmaceutical composition of claim 57 wherein the binding moiety is one member of a binding pair or a binding portion thereof selected from the group consisting of ligand-receptor, enzyme-substrate, and lectin-carbohydrate, and the other member of the binding pair is expressed on the pathogen.

72. The pharmaceutical composition of claim 57 wherein the binding moiety is an antibody or derivative thereof containing the binding domain.

73. The pharmaceutical composition of claim 63 wherein the binding moiety is an antibody or derivative thereof containing the binding domain.

74. The pharmaceutical composition of claim 67 wherein the binding moiety is an antibody or derivative thereof containing the binding domain.

75. The pharmaceutical composition of claim 68 wherein the binding moiety is an antibody or derivative thereof containing the binding domain.

76. A kit comprising in a container a purified particle bound to a binding moiety that specifically binds a pathogen, said binding moiety not being endogenous to said particle.

77. The method according to claim 1 which further comprises administering to the vertebrate an antibody that binds the particle.

78. The method according to claim 1 wherein said particle is opsonized with antibody to the particle.

79. A particle bound to a moiety that specifically binds to a pathogen, said binding moiety not being endogenous to said particle.
